(19) United States
(12) Patent Application Publication
(10) Pub. No.: US 2010/0292795 A1
(43) Pub. Date: Nov. 18, 2010

(54) BIOMEDICAL IMPLANT SURFACE TOPOGRAPHY

(76) Inventor: Ole T. Jensen, Englewood, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(21) Appl. No.: 12/779,376

(22) Filed: May 13, 2010

Related U.S. Application Data
(60) Provisional application No. 61/178,026, filed on May 13, 2009.

(51) Int. Cl. A61F 2/44; A61F 2/28; A61F 2/32; A61C 13/00; B23K 26/36; B05D 3/06

(52) U.S. Cl. 623/17.11; 623/16.11; 623/22.11; 433/167; 219/121.68; 427/2.26

(57) ABSTRACT

There is provided a method for creating a micro-topographical surface on biomedical implants that mimics a topography of bone to help facilitate osseointegration of the implant, as well as related devices and resulting implants. For example, a surface topography system is provided that includes a processor and a memory coupled to the processor. The processor is configured to process an image from a scanning electron microscope to determine a surface topography of bone. A surface manipulation device is configured to create the surface topography of bone on a surface of a biomedical implant.

### CROSS-REFERENCE TO RELATED APPLICATIONS

The present application is related to and claims priority to U.S. Provisional Patent Application No. 61/178,026, filed on May 13, 2009, entitled “Laser Imprinting of Bio Medical Implant Surface,” the contents of which are hereby incorporated by reference herein in their entirety.

### BACKGROUND

1. Technical Field

The present invention relates generally to biomedical implants and, more specifically, to creating a surface topography for biomedical implants conducive to osseointegration.

2. Background Discussion

Beyond being non-toxic, metals such as titanium and titanium alloys provide high strength, and are relatively light weight and corrosion resistant. As such, titanium and titanium alloys are commonly used in biomedical applications. For example, dental implants, joint replacement implants such as hip replacement implants, knee replacement implants, and so forth are commonly made of titanium and/or titanium alloys.

### BRIEF DESCRIPTION OF THE DRAWINGS

FIG. 1 is a flow chart generally depicting a process for creating a surface topography for an implant to help enhance bone attachment.

FIG. 2 is a block diagram of an imaging device for determining a topography of bone.

FIG. 3 is a block diagram of a laser or computer assisted oxidation system for creating a bone topography on an implant surface.

FIG. 4 illustrates a biomedical implant having a surface topography that approximates a bone surface topography.

FIG. 5 illustrates a cross sectional view of the surface of the implant of FIG. 4 along line AA after creating a surface topography to correspond to the bone surface topography.

FIG. 6 illustrates the implant surface of FIG. 5 with bone growth over the surface.

FIG. 7 illustrates a cross sectional view of a portion of an implant having an oxide that is deposited to generate an oxide topography over a surface of the implant that mimics bone surface topography to aid in osseointegration.

FIG. 8A illustrates a cross sectional view of a portion of an implant having an oxide layer deposited thereon.

FIG. 8B illustrates a cross sectional view of the portion of the implant of FIG. 8A after the oxide layer has been manipulated to mimic a surface topography of bone with surface lacunae.

FIG. 9 illustrates a cross sectional view of an implant surface having lacunae that are extended to form grooves.

### DETAILED DESCRIPTION

Embodiments set forth herein generally include providing biomedical implant surfaces, such as titanium and titanium alloy implant surfaces with a topography to help facilitate osseointegration of the biomedical implants such as bone replacements, hip implants, dental implants, etc. Generally, the topography of the surface mimics bone micro-topography to help facilitate osseointegration of the implant.

In some embodiments, a surface of a biomedical implant may be manipulated to facilitate osseointegration of the implant by removing material from the implant surface. Specifically, material is removed from the surface to create a topography that mimics or approximates a surface of bone in which osteoclast have removed material in accordance with a remodeling process. In some embodiments, the material may be removed using a computer guided laser. In other embodiments, the material may be removed using an etching process or other suitable process.

In some embodiments, a surface of a biomedical implant may be manipulated to facilitate osseointegration by adding material to the implant surface. For example, in some embodiments, an oxide layer, such as a titanium oxide, may be formed over the surface of the implant. In some embodiments, the oxide layer may be deposited on the surface of the implant such that it mimics or approximates a surface of bone in which resorption has occurred. Additionally or alternatively, the oxide layer may be modified by removal of portions of the oxide layer so that it mimics or approximates a bone surface where resorption has occurred in accordance with the biological mechanism of bone remodeling.

Generally, remodeling of bone tissue follows a specific activation, resorption, and formation (A-R-F) pattern. Activation refers to a process by which osteoclasts are recruited to a bone surface and signal coupling of osteoblasts. The osteoclasts resorb (i.e., remove) bone material to leave small depressions, grooves and/or apertures in the bone surface. The small depressions, grooves and/or apertures are believed to have a particular size, shape and spacing that facilitates recognition of the apertures by the osteoblasts. Osteoblasts fill the apertures and lay down bone structure. The formation stage proceeds in packets or units called BMU (Bone Metabolic Units), a process in which osteoblast cells are coupled in action in the formation of a new osteon. Thus, bone surface is first removed by the osteoclast and then osteoblasts enter in and bone formation commences. In this manner bone grafts are accepted and engrafted (consolidated) into the body by bone modeling (callus formation) followed by the A-R-F mechanism.

Imaging technology may be used to determine a surface topography of bone that has been resorbed during the A-R-F process. In particular, for example, scanning electron microscopy may be employed after demineralization of bone (in vivo and in vitro) by osteclasts, to obtain a micro-topographic image of the resorbed bone surface. The image may then be processed and analyzed to determine characteristics of the surface such as depth, circumference, shape and spacing of the apertures created by the osteoclasts in the surface. These characteristics may be provided to devices that configured to create the surface topology in a implant. For example, a computer guided laser may be implemented to replicates or nearly replicate the bone surface micro topology on a titanium or other implant surface.

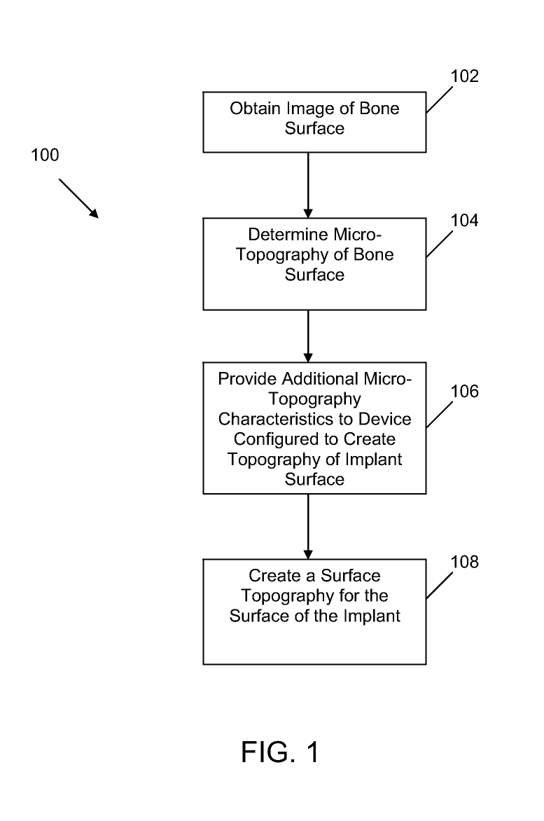

Turning to the figures and referring initially to FIG. 1, a flowchart illustrating a process 100 for creating a surface conducive to osseointegration of biomedical implants is illustrated. The process 100 begins by obtaining an image of the topography of the bone surface (Block 102). Generally, the image is used for the determination of bone surface topography and, as such, includes determining the topography of a bone surface where bone resorption has occurred. That is, a surface where activated osteoclasts have digested the bone to form lacunae. Suitable imaging technology may be implemented to obtain an image of the surface or otherwise determine the characteristics of the resorbed bone surface. For example, a scanning electron microscope may be used to obtain an image of the bone surface.

The image of the bone surface may be processed to determine characteristics of the surface micro-topography (Block 104). For example, the depth, width, shape and/or spacing of the lacunae may be determined. In some cases, the lacunae may be extended in the form of grooves having specific dimensions. One or more of the characteristics may be used to create the surface topography. In particular, one or more characteristics are provided to a device configured to create the topography on the surface of the implant (Block 106). In some embodiments, the image may be provided directly to a computer that interprets the images and controls the operation of a device configured to generate the topography. In other embodiments, a user may interpret an image and provide parameters to a computer or device to create a desired topology. In yet other embodiments, the images may be interpreted by the computer and parameters may be modified by the user to achieve a desired topology. Thus, in some embodiments, the surface topography may be determined automatically (e.g., by software executing on a computer system), while in other embodiments, a user may provide or modify the parameters that define the topography.

Once the parameters for the topography have been set by either a user or a computer, a surface topography of an implant may be generated according to the parameters (Block 108). In some embodiments, apertures may be made in the surface to mimic the lacunae of the bone surface. In some embodiments, the apertures may take the form of grooves within the surface of the implant that mimic lacunae of the bone surface.

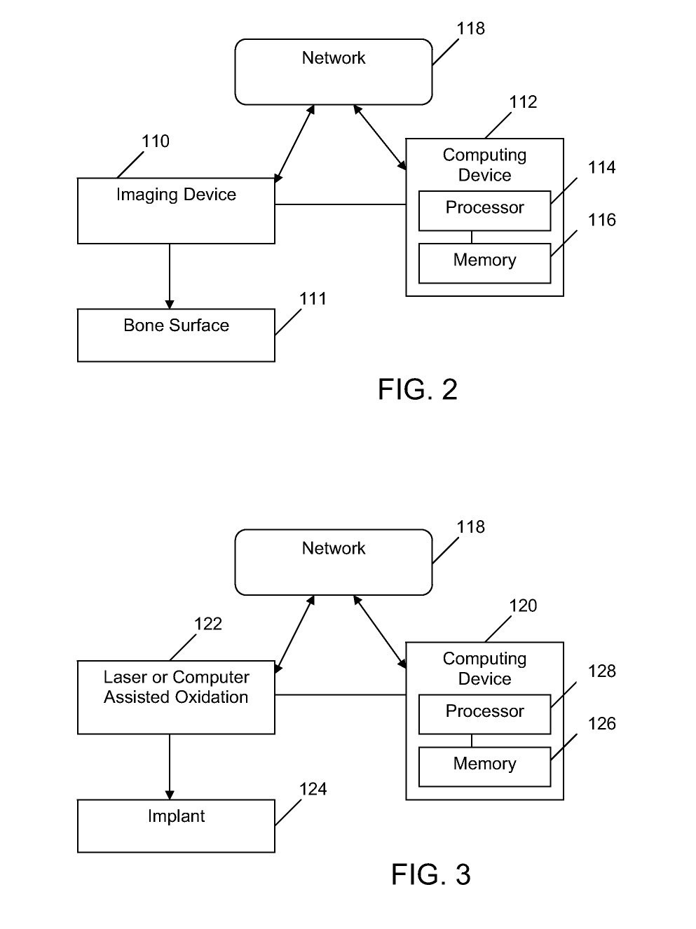

In other embodiments, layers may be provided on top of the surface that mimic the surface of a bone where resorption has occurred. For example, an oxide layer may be provided that mimics the resorbed bone. FIG. 2 illustrates an imaging device 110 configured to obtain an image of bone surface 111. The imaging device 110 may be any suitable device that is capable of capturing or generating images of objects on a nanometer scale. For example, in one embodiment, the imaging device 110 may be a scanning electron microscope.

Raw data obtained from the imaging device 110 may be processed and stored into a machine readable medium by a computing device 112 coupled to the imaging device 110. In some embodiments, the computing device 112 may be integral with the imaging device 110. Additionally, the imaging device 110 and/or the computing device 112 may be connected to a computer network 118 to help facilitate the transfer of data therebetween and to other computers.

The processing of the image and/or raw data may include filtering of the data to remove potential noise interference as well as digitizing of analog signals for processing, storage and reproduction. Additionally, the processing may include determining one or more characteristics of the bone's surface topography such as the size, depth, shape, spacing and/or arrangement of lacunae.

As such, the computing device 112 may include one or more processors 114 and storage devices 116 configured to operate software to provide such processing services. The processor 114 may be any suitable processor, microcontroller, or application specific controller available from a variety of manufacturers, including multicore processors available from Advanced Micro Devices (AMD) or Intel. The memory 116 may be any form of suitable computer readable medium, such as random access memory (RAM), dynamic RAM, static RAM, Flash, read only memory (ROM), hard disc drives, and so forth.

In some embodiments, the topography of the bone surface may be determined and templated for multiple subsequent uses. For example, the topography of jaw bone surface may be determined from a single sample and used to imprint multiple different implants. As such, software may be developed that utilizes the image of the bone surface to determine and/or create a pattern for use in generating an implant surface topography that is approximates or mimics the topography of the bone surface. The pattern may be used for implant surfaces that contact bone. In other embodiments, the topography of the bone surface may be determined (i.e., via scanning electron microscopy) for each implant and/or for each adjacent portion of bone where an implant will be positioned. That is, for each implant a topography of local bone surface is determined, as the bone topography may vary from patient to patient and/or between different sites of a single patient.

As discussed above, the image of the surface topography, parameters related to the surface topography, or both may be provided to a device configured to generate the surface topography in an implant surface. In some embodiments, the image or parameters may be provided to a computer coupled to a device for generating the surface topography in the implant. For example, as illustrated in FIG. 3, the image and/or parameters may be provided to a computing device 120 communicatively coupled to a surface manipulation device such as a laser or computer assisted oxidation system 122. The computing device 120 may be configured to precisely control the operation of the laser or computer assisted oxidation system 122. In particular, the computing device 120 is configured to receive the images and/or parameters and operate the laser or computer assisted oxidation system 122 to generate the surface topography in a surface of an implant 124. In some embodiments, the computing device 120 may be configured to autonomously read the image and/or parameters and operate the laser 122 to create the surface topology. In some embodiments, the computing device 120 may be configured to receive user input related to the topography, such as the parameters. The computing device 120 may be configured to receive the image, parameters and/or user input via a network, such as a network 118 coupled to the imaging device 110 and/or computing device 112.

The computing device 120 includes a memory 126 that may store operating instructions for the computing device and for the operation of the laser or computer assisted oxidation system 122. Additionally, the memory may store the topographical information for future reference and/or use. A processor 128 may be coupled to the memory 126 and configured to control the operations of the laser or computer assisted oxidation system 122 in accordance with programs stored in the memory. As such, the computing device 120 is configured to operate the laser or computer assisted oxidation system 122 to reproduce the stored topographical patterns on a surface of a biomedical implant. In an alternative embodiment, the computing device 120 may be integral to the laser or computer assisted oxidation system 122. In some embodiments, the computing devices 112 and 120 may be the same computing device. Additionally, it should be appreciated that in some embodiments the computing device 112 and 120 may be integral with the imaging device 110 or laser or computer assisted oxidation system 122, located proximately to the imaging device or laser and/or remotely located from the imaging device and laser.

The laser 122 may operate in any suitable wavelength range and at power levels suitable to imprint titanium, zirconium, or other material used for implants, as well as alloys of such metals and materials. The precise operating parameters may vary based on the material that is being imprinted and, therefore, may be determined empirically through laboratory testing. The laser 122 may be operated by the computing device 120 to imprint a topography onto an implant surface that mimics the topography of the bone surface.

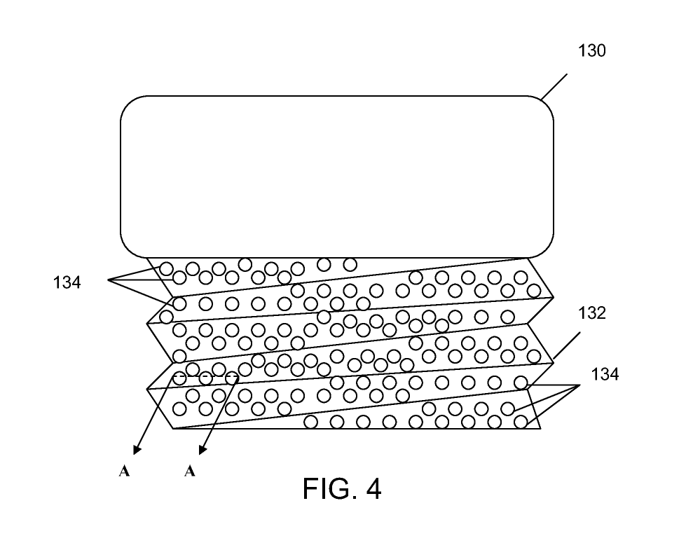

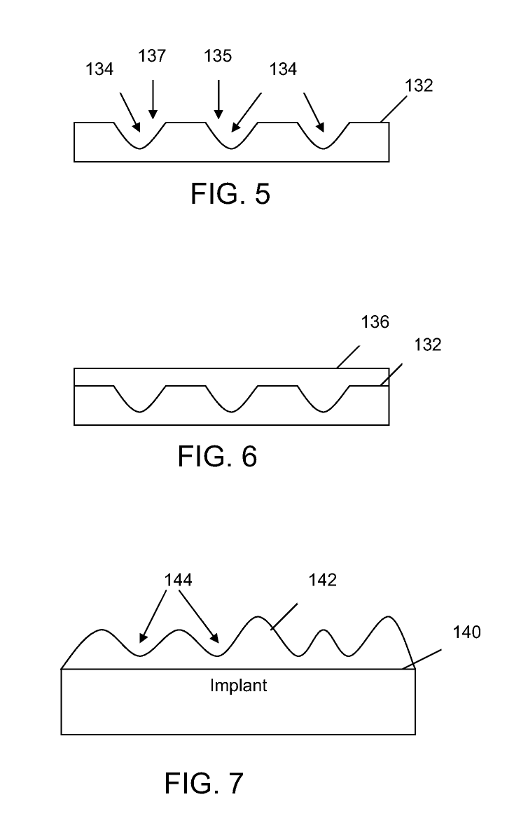

FIG. 4 illustrates an example implant 130 that has been laser imprinted with a surface micro-topography that mimics bone. Specifically, the surface 132 of the implant 130 has many apertures 134 or cavities that mimic osteoclast resorption lacunae. It should be appreciated that FIG. 4 and, indeed, all figures are not necessarily to scale and are intended to provide an understanding of certain features contained herein. In particular, for example, the apertures 134 illustrated in FIGS. 4 and 5 may not be properly scaled relative to the implant 130.

FIG. 5 is a cross-sectional view of the implant surface 132, showing the surface 132 and apertures 134 (as discussed above, the apertures 134 may mimic osteoclast resorption lacunae or grooves). Although little detail is illustrated on the surface 132 in FIG. 5, it should be understood that the surface imprinted by the laser 110 may mimic the bone surface in several, many or all microscopic dimensions including bone periodicity, the distance between osteons, reversal line micro-topography, collagen fibrils, etc. to form a highly osteoconductive surface for bone osteointegration. The implant when implanted into bone, though an oxidized metallic surface, becomes highly osteoconductive to bone annealing, at least in part due to the surface of the implant mimicking the bone surface.

Further, the surface 132 provides a baseline for nano-technological modification using nano-technological modification using nano-particles or nano-fibers. For example, the surface 132 may be further modified with nano-particles such as aluminum oxide nano particles, calcium phosphate nano particles, and so forth, that may be sprayed on or otherwise applied to the surface.

Referring again to the features illustrated in FIG. 5, the apertures 134 are spaced and have depths favorable for osteogenesis. Specifically, the apertures 134 (and/or osteons (not shown)) may have a periodicity of approximately 125 to 175 nm and depths of approximately 35-85 nm. For example, apertures 135 and 137 may be approximately 150 nm apart and aperture 137 may be 50 nm deep. Because the apertures spacing and depth mimic bone surface, the implant surface is recognized by adjacent cells as being bone. Hence, the implant surface is highly attractant to osteoblast cell attachment and subsequent mineralization. FIG. 6 illustrates bone 136 attachment to the surface 132 of the implant 120.

In some embodiments, an additional layer may be provided over the surface of the implant and the surface topography may be formed within the additional layer. For example, in some embodiments, an oxide layer, such as a titanium oxide layer may be formed over the surface of the implant.

As illustrated in FIG. 7, in some embodiments, the deposition of an oxide layer 142 may be precisely controlled such that it is deposited in a pattern that mimics or approximates the determined surface topology of bone. That is the oxide layer 142 is deposited on the surface 140 of an implant with apertures 144 that mimic the bone surface. In some embodiments, the oxide layer may have a crystalline structure and/or may be phosphate enriched such as the oxides implemented by TiUnite®. Although, it should be understood that the oxide layer 142 may have different structures and/or be enriched with other elements.

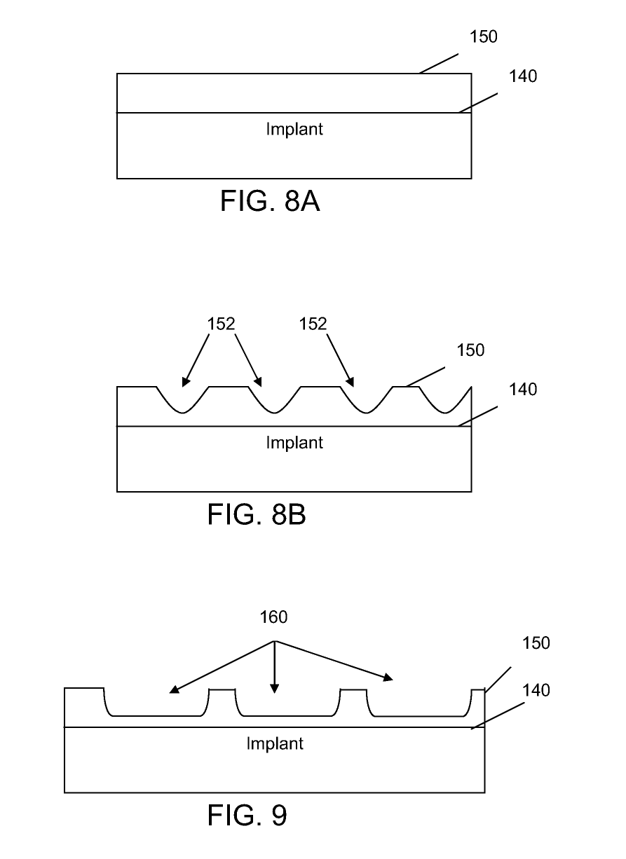

FIGS. 8A and 8B illustrate an alternative embodiment wherein, an oxide layer 150 is deposited on the surface 140 of the implant and subsequently the oxide layer may be manipulated to mimic or approximate the determined surface topography of the bone. For example, the oxide layer 150 may be etched using a laser or a chemical to create apertures 152 and other features to provide a desired topology, as shown in FIG. 8B.

FIG. 9 illustrates cross section of an implant having an oxide layer deposited thereon wherein the lacunae in the oxide layer 150 are formed as grooves 160 to mimic the characteristics of the lacunae in a bone surface. It should be appreciated that in some embodiments, the apertures may take one or more forms, including grooves, circles, or other geometric shapes. As such, some embodiments may include multiple different shaped apertures. Additionally, in should be appreciated that the grooves may be formed in the surface of the implant as well as in an oxide layer formed over the implant.

In accordance with the foregoing, implant surfaces may be provided that help enable osseointegration of the implant. In particular, imaging technology is used to generating a micro-topographic image of bone (in vivo and in vitro). The image may be implemented in replicating the micro topology on an implant surface so that it mimics the bone surface topographically. In some embodiments, the micro-topography of the implant may be manipulated through computer assisted processes to approximate bone surface. In particular, the surface topography of the implant includes favorable sites for osteogenesis, such as is found in osteoclast resorption lacunae, such that the implant is recognized as bone, rather than an implant and becomes highly attractant to osteoblast cell attachment and subsequent mineralization.

The foregoing surface treatment techniques have broad application in orthopedics for total joint replacement, including spinal implant surgery and in dentistry for dental implant osseointegration. Other example applications include, but are not limited to, elbow, knee, shoulder, hip, and ankle replacements, as well as other joints and bones located throughout the body. Additionally, although the technique has been described with respect to titanium and zirconium and their alloys, the creation of the surface topography may be performed on any bio implant material including ceramics, stainless, steel, plastics, or any other type of material to provide a surface conducive to bone growth and/or soft tissue attachment. Indeed, although the present subject matter has been described with respect to particular embodiments, it should be understood that changes to the described embodiments and/or methods may be made yet still embraced by alternative embodiments of the invention. Accordingly, the proper scope of the present invention is defined by the claims herein.

1. A surface topography system comprising:
a processor; and
a memory coupled to the processor, wherein the processor is configured to process an image from a scanning electron microscope to determine a surface topography of bone;
a surface manipulation device configured to create the surface topography of bone on a surface of a biomedical implant.

2. The surface topography system of claim 1 wherein the surface manipulation device comprises a laser configured to etch the surface of the biomedical implant.

3. The surface topography system of claim 1 wherein the surface manipulation device comprises a device for forming an oxide layer on the surface, the oxide layer being deposited on the surface in a pattern that approximates the surface topography.

4. The surface topography system of claim 1 wherein the surface manipulation device comprises a device for forming an oxide layer on the surface and subsequently etch the oxide layer to create the surface topography.

5. The surface topography system of claim 4 further comprising a laser to create the surface topography.

6. A laser imprinting system configured to read micro-topographical data from a computer readable medium, the micro-topographical data providing a micro-topography of bone, and control a laser beam to create micro-topographical pattern a surface of a biomedical implant that approximates the micro-topography of bone.

7. The computer guided laser imprinting system of claim 6 wherein the surface is one of a zirconium, zirconium alloy, titanium or titanium alloy.

8. The computer guided laser imprinting system of claim 6 comprising a processor communicatively coupled to the computer readable medium, wherein the processor is configured to execute instructions stored on the computer readable medium to control the laser beam.

9. The computer guided laser imprinting system of claim 6 wherein the computer readable medium stores computer executable instructions to read the micro-topographical data and operate the laser to imprint the topography on the surface.

10. A biomedical implant comprising a surface having a micro topography that mimics osteoclast bone surface for cellular attachment and proliferation.

11. The biomedical implant of claim 10 wherein the surface topography comprises a biologic periodicity of apertures of approximately 125-175 nm.

12. The biomedical implant of claim 10 wherein the surface topography comprises apertures that mimic osteoclast resorption lacunae having depths of approximately 35-85 nm.

13. The biomedical implant of claim 12 wherein the surface topography comprises features including at least one or more of the following: bone periodicity, a distance between osteons, reversal line micro-topography, and collagen fibrils.

14. The biomedical implant of claim 12 comprising a dental implant.

15. The biomedical implant of claim 12 comprising a spinal implant.

16. The biomedical implant of claim 12 comprising a hip implant.

17. A method of manufacturing biomedical implants comprising:
determining a micro-topography of bone; and
creating a surface of an implant that approximates the determined micro-topography.

18. The method of claim 17 comprising programming a computer operated laser to create the micro-topography on the surface of the implant.

19. The method of claim 17 comprising using a scanning electron microscope to determine the micro-topography of bone.

20. The method of claim 17 comprising depositing an oxide layer on the surface of the implant, the oxide layer approximating the determined micro-topography.
